(19) United States
(12) Patent Application Publication
(10) Pub. No.: US 2007/0293459 A1
(43) Pub. Date: Dec. 20, 2007

(54) Method for Inhibiting Cellular Na+-K+ ATPase Activity

(75) Inventors: Tzyy-Rong Jinn, Taichung (TW); Yi-Ching Chen, Taichung (TW); Tze-Cheng Tzen, Taichung (TW); Balance Chih-Ming Chen, Taichung (TW)

Correspondence Address: PAI PATENT & TRADEMARK LAW FIRM, 1001 FOURTH AVENUE, SUITE 3200, SEATTLE, WA, 98154, US

(73) Assignee: JASON LIFE TECH INC. LTD., Taichung (TW)

(21) Appl. No.: 11/551,348

(22) Filed: Oct. 20, 2006

Related U.S. Application Data
(63) Continuation-in-part of application No. 11/425,133, filed on Jun. 19, 2006.

(51) Int. Cl. A61K 31/58; A61K 31/555

(52) U.S. Cl. 514/171; 514/172; 514/185

(57) ABSTRACT

The present invention discloses an inhibitive effect of Na⁺—K⁺-ATPase caused by a compound selected from the group consisting of magnesium lithospermate B (MLB), isomer, prodrug, derivative, pharmaceutically acceptable salt, and a composition thereof. In this invention, the variations of Na⁺—K⁺-ATPase activity were monitored with increasing MLB concentrations, and the result shows the Na⁺—K⁺ ATPase activity is repressed by MLB. An outcome of the inhibitory effect, the function of cellular sodium/potassium exchanger is reduced and cellular calcium ion concentration is increased. The cerebral ischemia test exhibited MLB provides an effective repression of cell infarct. That is, the MLB is useful for inhibiting the function of cellular Na⁺—K⁺ pump, and further brings the utility for cardiac stimulation, diuretic enhancement, heart failure curing, anti-anoxia, neurocyte apoptosis protection, apoplexy prevention and treatment, and so on.

### CROSS-REFERRENCE TO RELATED APPLICATIONS

This application is a continuation-in-part of U.S. application Ser. No. 11/425,133, filed on Jun. 19, 2006, the disclosure of which is hereby incorporated by reference herein in its entirety.

### BACKGROUND

1. Field of Invention

The present invention relates to a phenomenon of Na⁺—K⁺ ATPase inhibitory activity of animal cells by treating with magnesium lithospermate B (MLB). More particularly, the present invention relates to the MLB is useful as therapeutic agent for purpose of cardiac stimulating, diuretic, apoplexy and the like.

2. Description of Related Art

In general, cardiac glycoside (such as ouabain and digoxin) is used to treat congestive heart failure (CHF), cardiogenic shock, and arrhythmia. The cardiac glycoside pharmacological mechanism inhibits cardiac cell membrane Na⁺—K⁺ ATPase activity and thereby represses the adenosine triphosphate (ATP) hydrolysis. Adenosine triphosphate (ATP) hydrolysis is essential for the cellular Na⁺—K⁺ exchanger. When the adenosine triphosphate (ATP) hydrolysis is repressed, the sodium and potassium ions that exchanging through the cell membrane are reduced. That results in the cellular Na⁺—Ca⁺ exchanger increases and rising the cellular Ca⁺ concentration. Consequently, the systole is enhanced and cardiac stimulation.

Recently, some research disclosed the cardiac glycoside or closely related compound provide positive effect for Ischemia-reperfusion of apoplexy, and the mechanism is believed to responsible for Na⁺—K⁺ ATPase inhibition. Another reference demonstrated the influence of Na⁺—K⁺ ATPase inhibition on apoptosis prevented by addition of a cardiac glycoside ouabain, specific inhibitor of the Na⁺ K⁺ ATPase. However, cardiac glycoside inhibits Na⁺—K⁺ ATPase activity and thereby suppresses the active Na⁺—K⁺ transport system of the cell. Therefore, when cardiac glycoside is taken in large quantities, the hyperkalemia might occurs frequently.

Cardiac glycoside has a narrow therapeutic index so it is difficult to determine between a therapeutic concentration and a poisonous concentration. The optimal cardiac glycoside dosage differs for each person wherein older people, heart disease patients or renal insufficiency patients are generally have a higher toxication risk. Therefore, the proper dosages of cardiac glycoside for each patient should be determined to prevent serious toxication.

Danshen (Salvia miltiorrhiza) is a traditional Chinese medicine used to stimulate blood circulation and to eliminate hematoma. A Chinese herbal medicine book, “Shennong's Classic of Materia Medica,” states Danshen is a highest-grade herb. A highest-grade herb is non-poisonous and can be used over a long-term. An active chemical of Danshen is magnesium lithospermate B (MLB or Salvianolic acid B, magnesium tanshinoate B), which has antioxidant properties. MLB benefits cell apoptosis and the regeneration of the intima to prevent the vascular intima from thickening. Moreover, Danshen also inhibits erythrocyte aggregation and increases the surface charge of erythrocyte membranes to protect the cardiovascular system. Danshen has been used in health care for thousand years in China.

### SUMMARY

The present invention is directed to a method to inhibit the cell membrane Na⁺—K⁺ ATPase activity with Danshen extract, magnesium lithospermate B (MLB), and satisfies the need for an alternative medicine differing from cardiac glycoside without the danger of toxication.

According to the preferred embodiment, the variations of Na⁺—K⁺-ATPase activity of cortex and cardiac cell membrane were monitored with increasing MLB concentrations. The result shows that Na⁺—K⁺ ATPase activity is repressed by MLB, which extracted from its source plant Danshen. The proportion phenomenon can determine that MLB is a Na⁺—K⁺ ATPase inhibitor.

The preferred embodiment of the present invention, discloses MLB provides a mechanism similar to cardiac glycoside. The Na⁺—K⁺ ATPase activity is depressed when treated with various MLB concentrations and compared to cardiac glycoside, ouabain, and the inhibitory behavior of MLB is consistent with the ouabain.

Another preferred embodiment of the present invention, discloses MLB provides positive effects of against cerebral hypoxia and cerebral infract in lower dosage. Moreover, the lower dosage MLB has significant repress the cerebral infract volume and size under a focal cerebral ischemia model. The result shows the MLB has a purpose to prevent and treat for the cerebral infract caused by ischemia-reperfusion of ischemia stroke.

That is, MLB is able to repress the Na⁺—K⁺ ATPase activity and is useful for cardiac stimulation, diuretic enhancement, heart failure cures, anti-anoxia, neurocyte apoptosis protection, apoplexy prevention and treatment, and so on. Mechanisms of above-mentioned effects are relative to the inhibition of Na⁺—K⁺ ATPase activity.

Consequently, MLB is an equivalent to the cardiac glycoside and contributes to an alternative medicine different from cardiac glycoside to develop a pharmaceutical agent or functional food.

It is to be understood that both the foregoing general description and the following detailed description are by examples, and are intended to provide further explanation of the invention as claimed.

### BRIEF DESCRIPTION OF THE DRAWINGS

These and other features, aspects, and advantages of the present invention will become better understood with regard to the following description, appended claims, and accompanying drawings where:

FIG. 1 is a diagram of measured Na⁺—K⁺ ATPase activity of a rat brain cortex treated with various MLB concentrations.

FIG. 2 is another diagram of measured Na⁺—K⁺ ATPase activity of a rat brain cortex treated with various MLB or ouabain concentrations.

FIG. 3 shows two molecular structures of MLB and cardiac glycoside.

FIG. 4 is a diagram of measured Na⁺—K⁺ ATPase activity of a rat brain cortex treated for various MLB concentrations.

FIG. 5 is another diagram of measured Na⁺—K⁺ ATPase activity of a rat myocardium cell membrane treated for various MLB concentrations.

FIG. 6 is a diagram shows the rat survival rate test by feeding sodium nitrite.

FIG. 7 is a diagram of cell infract preventive ability test by treating MLB after ischemia-reperfusion operation.

FIG. 8 is a diagram of cure rate test of cell infract by treating MLB after ischemia-reperfusion operation.

### DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

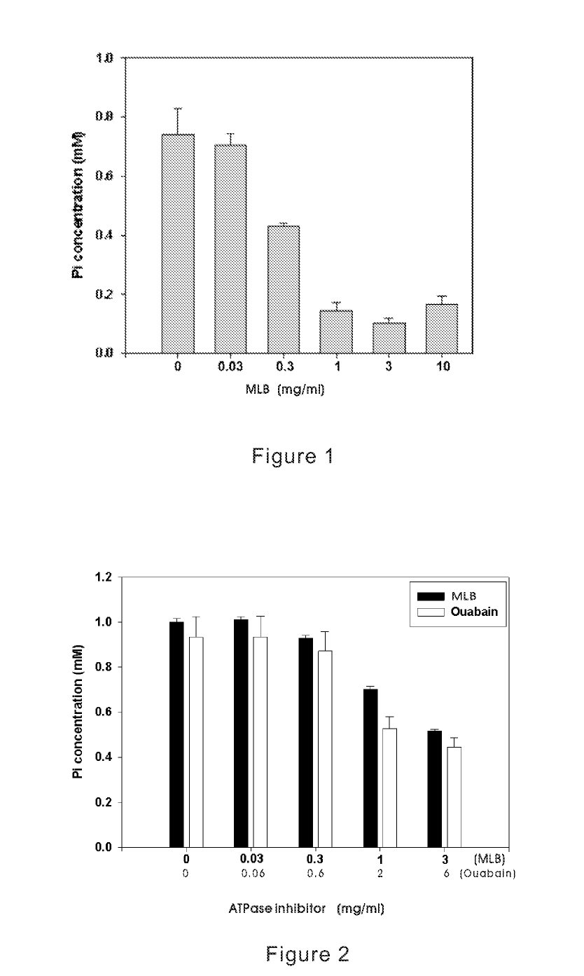

In the present invention, the action of MLB as an innovative cellular Na⁺—K⁺ ATPase inhibitor is disclosed. FIG. 1 is a diagram of measured Na⁺—K⁺ ATPase activity of rat brain cortex treated for various MLB concentrations, which shows the inhibitory effect of increasingly MLB concentration. Results are determined using the following steps:

First, Male Sprague-Dawley (NarII: SD) rats (3-month-old) were purchased from National Laboratory Animal Center (Nankang, Taipei) and raised under specific pathogen-free conditions. Animals were provided with rat chow (Rodent Laboratory Chow 5001, Purina, Mo.) and tap water throughout the studies. The rats received humane care in accordance with the guidelines of a guidebook for the care and use of laboratory animals. The animals were sacrificed by decapitation, and the brain and heart organs of the rats were removed immediately after complete exsanguination.

Next, the plasma membrane was isolated from the rat brain and heart at 4° C. The brain and heart homogenate were prepared respectively with homogenized plasma membrane in 10-20 volumes of 0.32 mM sucrose solution containing 5.0 mM 4-(2-hydroxyethyl)-1-piperazineethanesulfonic acid (HEPES) and 1.0 mM EDTA, pH7.5. Then, the brain or heart homogenate was centrifuged at 1000×g for 10 min, and kept the resultant supernatant for further centrifuge at 17000×g for 30 min to obtain a crude plasma membrane fraction.

Then, the fraction was washed and suspended twice in 0.32 M sucrose HEPES-buffer, which was subjected to a discontinuous sucrose density gradient consisting of successive layers of 0.3, 0.8 and 1.0 mM, and centrifuged at 63000×g for 1 hour. The plasma membrane was collected at the interface between 0.8 and 1.0 mM sucrose to be further suspended in 0.32 M sucrose solution for enzyme assays within 2 hours.

Na⁺—K⁺ ATPase activity was determined by measuring the amount of inorganic phosphate (Pi) liberated from ATP hydrolysis.

First, commercial Na⁺—K⁺ ATPase from porcine cerebral cortex (Sigma, 0.3 units/mg) or a purified plasma membrane fraction incorporated into a reaction mixture of 1 ml containing 3 mM ATP, 5 mM MgCl₂, 80 mM NaCl, 20 mM KCl, and 40 mM Tris-HCl, pH7.4, was prepared. The enzymatic reaction was terminated 15 min after Na⁺—K⁺ ATPase incorporation by adding 200 μl of 30% (WN) trichloroacetic acid.

After centrifugation at 6000 rpm for 15 min, supernatant of 500 μl measured the inorganic phosphate using spectrophotometric methods. Enzyme activity was expressed as μmol Pi liberated from ATP by 1 mg of Na⁺—K⁺ ATPase during 1 hour. Protein content was quantified using a Bradford protein assay kit (Sigma). For the observation of inhibitory effects on Na⁺—K⁺ ATPase activity, ouabain or MLB of various concentrations was incubated with commercial Na⁺—K⁺ ATPase or the purified plasma membrane fraction at 37° C. for 10 min prior to incorporation into the reaction mixture.

Reference is made to FIG. 1, which shows the inhibitory effect of Na⁺—K⁺ ATPase activity with increasing MLB concentration. The X-axis represents the variations of MLB concentration incorporated with the reaction mixture, and the Y-axis represents the variations amount of inorganic phosphate (Pi) liberated from ATP hydrolysis. As shown as FIG. 1, the measured free inorganic phosphate (Pi) decreases with increased MLB concentration showing the ATPase inhibition effect is dependent on raised MLB dosage. According to the proportion phenomenon the MLB is a Na⁺—K⁺ ATPase inhibitor.

The results of FIG. 1 disclose MLB provides a mechanism similar to the cardiac glycoside. FIG. 1 shows MLB represses the Na⁺—K⁺ ATPase activity. Accordingly, the Na⁺—K⁺ ATPase activity is depressed when treated with different MLB concentration. MLB activity Na⁺—K⁺ ATPase activity depression is compared with a cardiac glycoside, ouabain. As shown in FIG. 2, the inhibitory behavior of MLB is consistent with the ouabain, and the amount of free inorganic phosphate (Pi) is dependent on the raised inhibitor (MLB or ouabain) dosage.

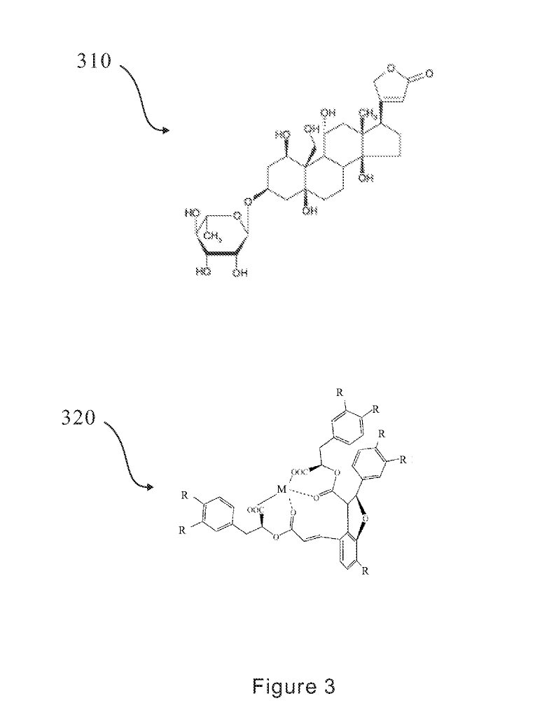

Reference is made to FIG. 3, which shows the molecular structures of the MLB and a cardiac glycoside. The molecular structure of cardiac glycoside 310 consists of a steroid core and at least one glycoside group. The molecular structure of MLB 320 is a compound with a metal ion located in the center of the MLB structure. In consideration, according to the results shown in FIG. 2, the inhibitory behavior of MLB is consistent with ouabain, and the enzyme activation is a “key and lock” model. MLB conformation plays a similar role as cardiac glycoside in inhibiting Na⁺—K⁺ ATPase activity. That is, MLB conformation is similar to cardiac glycoside and inhibits the Na⁺—K⁺ ATPase activity by way of inter-molecule affinity (secondary bond), such as electrostatic bond, hydrogen bond, hydrophobic bond or van der Waals bond.

According to the preferred embodiment of the present invention, the central MLB metal ion is a two-valence metal cation, such as magnesium, iron, manganese, calcium, zinc, copper or cobalt. Furthermore, the functional group (side chain) “R” of the MLB 320 comprises hydrogen, hydroxyl group, alkane, alkene, alkyne, aromatic group, glycosyl group or combined thereof.

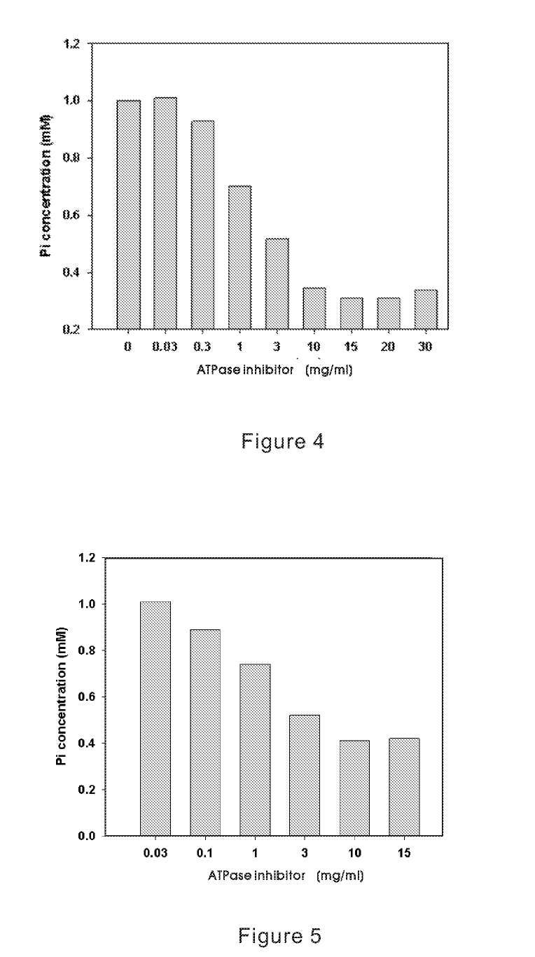

References are made to FIG. 4 and FIG. 5, which show the inhibitory effect of the Na⁺—K⁺ ATPase activity of MLB in cardiac and cortex cell membrane, respectively. FIG. 4 is the result of Na⁺—K⁺ ATPase activity of brain cortex cells treated with various MLB concentrations. The X-axis represents the variations MLB concentration incorporated with the reaction mixture, and the Y-axis represents the variations amount of inorganic phosphate (Pi) liberated from ATP hydrolysis. The result of FIG. 4 shows the measured free inorganic phosphate (Pi) decreasing with increased MLB concentration showing Na⁺—K⁺-ATPase activity is repressed by MLB, and the ATPase inhibition effect is dependent on raised MLB dosage. Hence, the MLB is able to inhibit the Na⁺—K⁺ ATPase activity of the cortex cell membrane. FIG. 5 is another result of Na⁺—K⁺ ATPase activity of cardiac cells membrane treated with various MLB concentrations that shows a consistent result with FIG. 4. The X-axis represents the variations in MLB concentration incorporated with the reaction mixture, and the Y-axis represents variations in the amount of inorganic phosphate (Pi) liberated from ATP hydrolysis. The result of FIG. 5 shows the measured free inorganic phosphate (Pi) decreases with increased MLB concentration indicating Na⁺—K⁺-ATPase activity is repressed by MLB, and the ATPase inhibition effect is dependent on raised MLB dosage. Hence, MLB is able to inhibit the Na⁺—K⁺ ATPase activity of cardiac cell membranes.

According to the results of FIG. 1-5, the MLB is able to inhibit the Na⁺—K⁺ ATPase activity of cardiac cell membrane, and therefore bring the physical effects to reduce the function of the cellular sodium/potassium exchanger and increases cellular calcium ion concentration. For this reason, the MLB provides an identical mechanism as the cardiac glycoside, that is MLB stimulates the systole for the purpose of cardiac stimulation, diuretic enhancement and another purpose of diuretic enhancement.

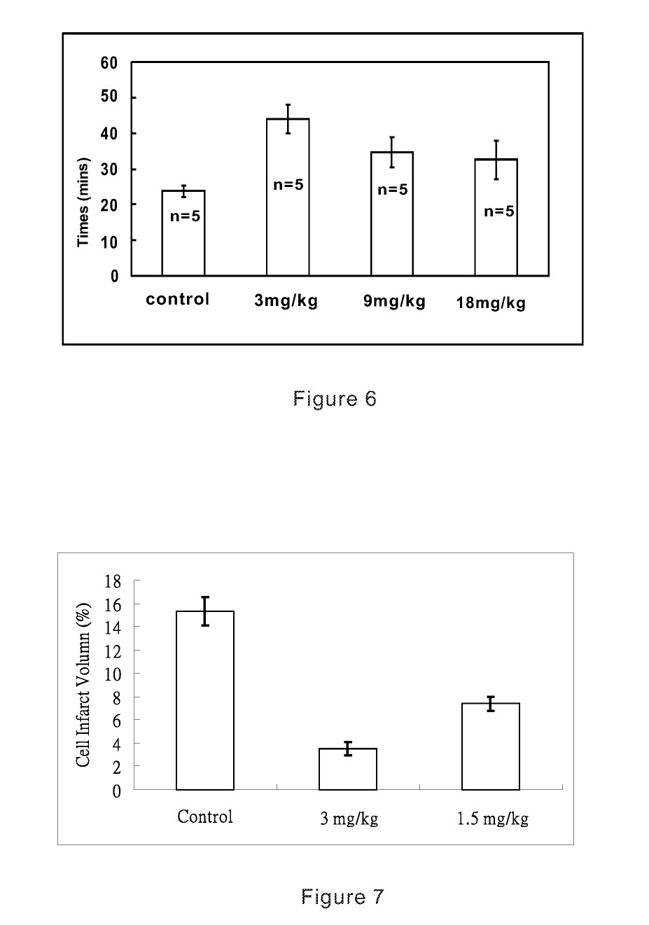

Furthermore, FIG. 6 shows the rat survival rate test by feeding sodium nitrite to test the preventive effect of MLB for cerebral hypoxia. Sodium nitrite will trigger the oxyhemoglobin converts into methemoglobin to cause the hypoxia and cell infract.

3-4 week old rats (about 20˜22 g) were randomly divided into three sample groups and one control group, and each group has 5 rats. Each group was oral fed with saline (control) or MLB (3 mg/kg, 9 mg/Kg, or 1.5 mg/Kg) respectively. After feeding for 7 days, one hour later of the final feeding, each rat was injected with sodium nitrite (225 mg/kg). Then count time on the spot to record the survival time of rats.

As shown as FIG. 6, the survival rate of group of injected 3 mg/Kg with MLB shows a significant (p<0.05) increase (two folds) as compared with the control group. The result exhibited the survival rate boosted by MLB after sodium nitrite fed. Also, the survival rate of group of injected 9 mg/Kg with MLB and 18 mg/Kg with MLB show significant (p<0.05) different from the control group. Accordingly, the MLB provides preventing effect for cerebral hypoxia and postponing the cerebral cell apoptosis.

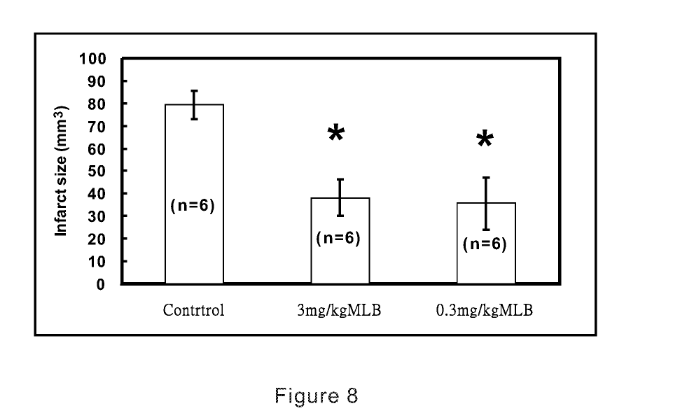

References are made to FIG. 7 and FIG. 8, which show infarct preventive ability test by treating MLB after ischemia-reperfusion operation. The test is used to imitate the cerebral infarct of stroke model. 15˜18 male gerbils (60-85 g) were randomly divided into three groups fed by regular meal supplemented (op., 20 mg/kg/day).

After feeding for 4 days, each gerbil was anesthetized with chlorohydrate (400 mg/kg) intraperitoneally and its body temperature was maintained at 37° C. with a heating pad (CMA/150). A midline neck incision was made and the right carotid artery was exposed and separated from the vago-sympathetic trunk. The gerbil's head was placed in a stereotaxic frame (David Kopf, CA, USA) with the nose bar positioned 4.0 mm below the horizontal line. Following a midline incision, the skull was partially removed to expose the right middle cerebral artery. The middle cerebral artery was loosely encircled with an 8-0 suture for later occlusion.

A focal cerebral ischemia was induced by occlusion of the right common carotid artery (CCA) and the right middle cerebral artery (MCA) for 60 min, followed by reperfusion for 3 h. MBF 3D, a laser probe (0.8 mm in diameter) of a Laser Doppler Blood Flow monitor (Moor Instruments, Axminster, England) was positioned onto the cortex with its tip close to the middle cerebral artery. Cerebral blood flow dropped to less than 5% of basal after the occlusion of the MCA. Cerebral blood flow reached its minimal level within 5 min after the start of the occlusion and was confirmed to remain at this level throughout the monitoring period to ensure the validity of the stroke model.

Approximately 24 h after cerebral ischemia, each gerbil was anesthetized and perfused transcardially with 2% isotonic heparinized saline and 2,3,5-triphenyltetrazolium chloride (TTC). A right reflex measurement is carried out prior to the perfusion. The brain was then removed and sliced into five or six slices of 2-mm-thick coronal sections for TTC staining. The brain slices were placed in 10% buffered formalin in the dark and then refrigerated until photographed.

Infarct size was quantified by weighing the traced normal and infracted areas. All TTC data were analyzed by ANOVA with Student t tests, and P<0.05 was considered to be statistically significant.

Referring to FIG. 7, thirty minutes before starting reperfusion, each group gerbil was injected intraperitoneally with saline (control) or MLB (3 mg/Kg and 1.5 mg/Kg) respectively. The cell infarct volume of group of injected 3 mg/Kg with MLB shows a significant (p<0.01) difference from the control group. Also, the group of injected 1.5 mg/Kg with MLB shows an effective repression of cell infarct.

Referring to FIG. 8, thirty minutes after reperfusion, each group gerbil was forced oral fed with saline (control) or MLB (3 mg/Kg and 0.3 mg/Kg) respectively. The cell infarct size of group of injected 3 mg/Kg with MLB shows a significant (p<0.01) difference from the control group. Also, the group of injected 0.3 mg/Kg with MLB shows an effective repression of cell infarct.

The above-mentioned summary the MLB provides a positive effect for Ischemia-reperfusion of apoplexy, and the mechanism is similar to the cardiac glycoside. Accordingly, the MLB also has a benefit for apoplexy prevention and treatment.

In accordance with the preferred embodiment of the present invention, the MLB is applied to treat disease selected from a group consisting of congestive heart failure (CHF), arrhythmia (such as atrial fibrillation, atrial flutter, and paroxysmal tachycardia), hypertension, edema, coronary heart disease (such as angina pectoris and myocardial infarction), anti-anoxia, neurocyte apoptosis protection, apoplexy prevention and treatment, and diseases related to the foregoing disease.

Consequently, an effective dosage of MLB provides a utility for cardiac stimulation and diuretic enhancement that is equivalent to the cardiac glycoside mechanism and contributes to an alternative medicine different from cardiac glycoside to develop a pharmaceutical agent or functional food. For example, it can be used to produce an active pharmaceutical ingredient or dietary supplement

In accordance with the preferred embodiment of the present invention, the structure shown in FIG. 3, named MLB 320, represents the MLB and derivatives thereof. Wherein, the MLB and derivatives thereof comprise the isomer, prodrug, and pharmaceutical acceptable salt thereof.

In another preferred embodiment of the present invention, a composition that comprise the compound structure of MLB 320 as an active principal is used to repress the cell membrane's Na⁺—K⁺ ATPase activity. Wherein, the active principal of the composition comprises of pharmaceutically acceptable salt, solvate, solvate of the pharmaceutically acceptable salt, polymorphism, and a prodrug of the MLB 320. Furthermore, the composition further comprises a pharmaceutical/food acceptable carrier, such as pharmaceutical/food acceptable assisting agent, thinner, excipient, or combination thereof. The MLB and the original herb “Danshen” and extract thereof, can be used to produce an active pharmaceutical ingredient or dietary supplement.

Moreover, the above-mentioned has shown the inhibitory effect of MLB on Na⁺—K⁺ ATPase activity. The cardiac stimulation and diuretic enhancement function of MLB and derivates thereof are equivalent to the cardiac glycoside. The present invention discloses that MLB is an alternative medicine differing from cardiac glycoside without the danger of toxication.

Although the present invention has been described in considerable detail with reference to certain preferred embodiments thereof, other embodiments are possible. Therefore, the spirit and scope of the appended claims should not be limited to the description of the preferred embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A method to inhibit cellular Na⁺—K⁺ ATPase activity comprising of:
administering a magnesium lithospermate B (MLB) and derivatives thereof to animal cells, which represented by following formula: 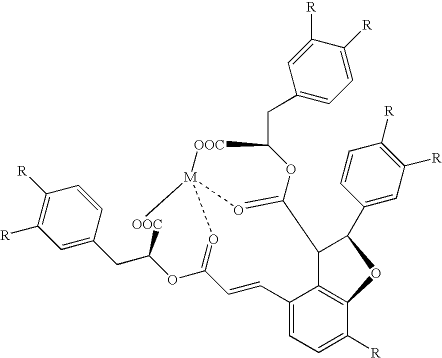
wherein the “M” represents a metal ion, the “R” represents any functional group; and
inhibiting adenosine triphosphate (ATP) hydrolysis, which is essential for cellular Na⁺—K⁺ exchanger, by MLB and derivatives thereof.

2. The method of claim 1, wherein the metal cation comprises magnesium, iron, manganese, calcium, zinc, copper or cobalt.

3. The method of claim 1, wherein the functional group comprises hydrogen, hydroxyl group, alkane, alkene, alkyne, aromatic group, glycosyl group or combined thereof.

4. The method of claim 1, wherein the MLB derivatives comprise a isomer, prodrug, pharmaceutically acceptable salt, and composition thereof.

5. The method of claim 4, wherein the pharmaceutically acceptable salt comprises magnesium salt, potassium salt, ammonium salt, or calcium salt.

6. The method of claim 1, wherein the ATP hydrolysis repressed by administering MLB and derivatives thereof as an effective dosage sufficient for inhibiting the Na⁺—K⁺ ATPase activity.

7. A composition for repressing the cell membrane's Na⁺—K⁺ ATPase activity, which comprises the compound structure cited in claim 1 as an active principal.

8. The composition of claim 7, wherein the active principal comprises pharmaceutical acceptable salt, solvate, solvate of the pharmaceutical acceptable salt, polymorphism, and prodrug of said compound.

9. The composition of claim 7, wherein the composition further comprises a pharmaceutical/food acceptable carrier.

10. The method of claim 9, wherein the pharmaceutical/food acceptable carrier comprises pharmaceutical/food acceptable assisting agent, thinner, excipient, or combination thereof.

11. The composition of claim 7, wherein the composition is original herb Danshen, and extract thereof.

12. The composition of claim 7, wherein the composition is an active pharmaceutical ingredient.

13. The composition of claim 7, wherein the composition is a dietary supplement.

14. The composition of claim 7, wherein the composition is a cardiac stimulation agent.

15. The composition of claim 7, wherein the composition is an anti-anoxia agent.

16. The composition of claim 7, wherein the composition is a neurocyte apoptosis protection agent.

17. The composition of claim 7, wherein the composition, the original herb Danshen of the active principal is applied to treat diseases selected from a group consisting of:
a) Congestive heart failure (CHF);
b) Arrhythmia, which comprise atrial fibrillation, atrial flutter, and paroxysmal tachycardia;
c) Hypertension;
d) Edema;
e) Coronary heart disease, which comprise angina pectoris, myocardial infarction and diseases related to the foregoing disease; and
f) Apoplexy.

18. A pharmaceutical derivative with a steroid structure as a core, characterized in another compound substitute for the core, wherein the compound is represented by the following formula:

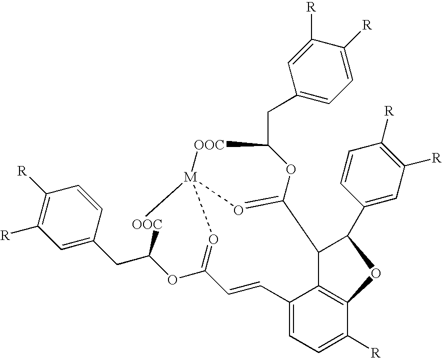

19. The method of claim 18, wherein the “M” represents a metal ion, and the “R” represents any functional group.

20. The method of claim 19, wherein the metal ion comprises two-valence metal cation.

21. The method of claim 19, wherein the functional group comprises hydrogen, hydroxyl group, alkane, alkene, alkyne, aromatic group, or combined thereof.
